(19) United States
(12) Patent Application Publication
(10) Pub. No.: US 2026/0295036 A1
(43) Pub. Date: Oct. 1, 2026

(54) VACCINE FOR TREATING ALLERGIES

(71) Applicant: WORG PHARMACEUTICALS (ZHEJIANG) CO., LTD, Huzhou, Zhejiang (CN)

(72) Inventors: Alexander KARAULOV, Huzhou, Zhejiang (CN); Rudolf VALENTA, Huzhou, Zhejiang (CN); Petr V. GLYBOCHKO, Huzhou, Zhejiang (CN); Antonina KARSONOVA, Huzhou, Zhejiang (CN); Ksenja RIABOVA, Huzhou, Zhejiang (CN); Daria TRIFONOVA, Huzhou, Zhejiang (CN); Mirela CURIN, Huzhou, Zhejiang (CN); Marianne Van HAGE, Huzhou, Zhejiang (CN)

(21) Appl. No.: 19/127,971

(22) PCT Filed: Nov. 7, 2022
(86) PCT No.: PCT/CN2022/130414

§ 371 Date: May 7, 2025

(51) Int. Cl. A61K 39/35; A61K 39/00; A61P 37/08; C07K 14/47

(52) U.S. Cl. CPC A61K 39/35; A61P 37/08; C07K 14/47; A61K 2039/545; C07K 2319/40

(57) ABSTRACT

A peptide consisting of 20 to 30 amino acid residues derived from amino acid position 120 to 171 of mature allergen Fel d 4 is provided as well as fusion proteins comprising said peptide. A vector includes a nucleic acid molecule encoding the peptide. A vaccine formulation includes the peptide is effective in prevention or treatment of a furry animal allergy.

### TECHNICAL FIELD

The present invention relates to the field of allergies and means and methods for treating allergies.

### BACKGROUND ART

Furry animals, in particular cats, represent one of the most important indoor allergen sources in most parts of the world. Allergic patients sensitized to furry animals suffer from a variety of allergic symptoms, including respiratory allergy symptoms such as rhinitis and asthma, conjunctivitis and different manifestations of dermatitis.

Regarding cat allergy, Fel d 1 is the most important allergen which shows high IgE reactivity in the majority of cat-sensitized patients and high allergenic activity. It is a major target for IgE antibodies in patients suffering from respiratory allergy to cat and it has been shown that children suffering from cat-related symptoms of asthma are strongly sensitized to Fel d 1. Accordingly, the development of modern molecular allergen-specific forms of treatment has mainly focused on Fel d 1.

One of these molecular approaches for treatment cat allergies is the use of Fel d 1-derived hypoallergenic T cell epitope-containing peptides for allergen-specific immunotherapy (AIT) which has been evaluated in the first clinical trials using a limited number of Fel d 1 peptides (PS Norman, Annals of Allergy, 71(1993):330-3). This approach was then further developed by including additional peptides to achieve broad coverage of MHC class II diversity in patients (M Worm et al. J. Allergy Clin. Immunol. 127(2011):89-97). Other molecular approaches for allergen-specific immunotherapy of cat allergy also only focused on the major cat allergen Fel d 1 (K Niespodziana et al. J Allergy Clin Immunol. 127(2011):1562-70). Other allergen-specific treatment approaches are based on passive immunization with Fel d 1-specific monoclonal IgG antibodies which block allergic patients IgE binding to Fel d 1 (MA Kamal et al. Clin Transl Sci. 2021; 14:2440-2449).

However, so far allergen-specific therapeutic approaches have so far not considered the possible relevance of IgE sensitization to cat allergen molecules other than Fel d 1, and in particular not those which may show IgE cross-reactivity to allergens of other furry animals such as Fel d 4 and Fel d 7 and Can f 6. No allergen-specific approach for the combined treatment and prevention of allergy to several furry animals has been described so far.

Hence, it is an object of the present invention to provide methods and means to address this problem.

### SUMMARY OF THE INVENTION

The present invention relates to a peptide consisting of 20 to 30 amino acid residues derived from amino acid position 120 to 171, i.e. the C-terminal end, of mature allergen Fel d 4.

It turned surprisingly out that the peptide as defined herein is able to induce the formation of antibodies in a human or mammalian body which inhibit the binding of Fel d 4 specific IgEs of allergic patients to allergens. The prevention or reduction of allergen-IgE binding results in the prevention or alleviation of allergy symptoms caused by the respective allergen (i.e. cat allergen, in particular Fel d 4). It was unexpectedly found that other fragments derived from of Fel d 4, which are derived from amino acid position 1 to 119 of the mature allergen are not able to induce the formation of IgE blocking antibodies. This property of the inventive peptides can be used for the production of vaccines for treating and/or preventing cat allergies in humans and mammals.

In addition to the formation of Fel d 4 specific antibodies, the peptides of the present invention are also capable to induce the formation of antibodies directed to other allergens derived from furry animals other than cats (e.g. horses). It is a surprising finding that the antibodies induced by the peptides of the present invention show binding to allergens of other furry animals. Hence, the peptides of the present invention can also be used in the prevention and/or treatment of allergies caused by furry animals in general and in particular caused by cats, horses and dogs.

Another aspect of the present invention relates to a fusion protein or a conjugate comprising at least one peptide according to the present invention.

In order to enhance the immune response caused by the administration of the peptides of the present invention, for instance, these peptides can be part of fusion proteins and conjugates. Furthermore, the peptides of the present invention may also be fused or conjugated to other allergens and/or allergen fragments of the same source (i.e. cat) or from any other allergen sources. This allows to obtain fusion proteins and conjugates which can be used in the treatment of allergies triggered, for instance, by pollen, animal dander, dust mites, mold, food like peanuts, tree nuts, wheat, soy, fish, shellfish, eggs and milk or insect stings, such as from a bee or wasp.

A further aspect of the present invention relates to a peptide or a fusion protein or a conjugate according to the present invention for the use in the prevention or treatment of cat allergy.

The peptides, fusion proteins and conjugates of the present invention induce the formation of allergen specific antibodies. These antibodies, which typically include IgG, bind to allergens to which a subject (human or mammalian) is exposed. This binding prevents that these allergens bind to allergen specific IgE causing an allergic reaction.

Another aspect of the present invention relates to a peptide or a fusion protein or a conjugate according to the present invention for the use in the prevention or treatment of a furry animal allergy, preferably a dog or horse allergy.

It turned surprisingly out that the peptides, fusion proteins and conjugates of the present invention not only induce the formation of antibodies directed to a single allergen but also directed to other allergens. This cross reactivity is particularly advantageous because it allows to use that the peptides, fusion proteins and conjugates of the present invention also in the treatment of furry animals in general.

A further aspect of the present invention relates to a nucleic acid molecule encoding a peptide or a fusion protein according to the present invention.

Another aspect of the present invention relates to a vector comprising a nucleic acid molecule according to the present invention.

An aspect of the present invention relates to a host cell comprising a nucleic acid molecule or a vector according to the present invention.

A further aspect of the present invention relates to a vaccine formulation comprising a peptide, a fusion protein, a nucleic acid molecule and/or a vector according to the present invention.

### BRIEF DESCRIPTION OF THE FIGURES

FIG. 1 shows the prevalence of IgE reactivity and IgE levels to cat allergens in a Russian and Swedish population of cat allergic patients by ImmunoCap (kUA/L).

FIG. 2 shows inhibition of patients IgE binding to allergens obtained with anti-peptide antisera sera compared to antisera raised against the complete allergens (see Example 2).

FIG. 3 shows a combination of antisera against P3 and P5 from Fel d 7 shows better inhibition of patients IgE binding than antisera raised against the individual peptides.

FIG. 4 shows IgG antibodies raised against peptides from Fel d 7 and Fel d 4 prevent IgE binding to homologous allergens from dog (Can f 1) and horse (Equ c 1), respectively.

FIG. 5 shows PreS fusion proteins (labeled “SuperCats”) containing two copies of the Fel d 1 (P1, P5), Fel d 4 (P9) and Fel d 7 (P3, P7) derived peptides in different order.

FIG. 6 shows a comparison of the IgE binding capacity of SuperCats 1 to 5 with equimolar mixtures of Fel d 1, Fel d 4 and Fel d 7 by ImmunoCap measurements.

FIG. 7 shows a comparison of the allergenic activity of SuperCats and mix of cat allergens in RBL assay.

FIG. 8 shows a comparison of all SuperCat constructs in competitive ELISA.

FIG. 9 shows FIG. 9 a comparison of the capacity of antibodies obtained by immunization with SuperCats 1, 3 and 5 and previous Fel d 1-based vaccines PreS-P1-P5 and PreS-2×P1 to inhibit allergic patients (n=11) IgE binding to Fel d 1.

FIG. 10 shows a protocol of rabbit immunizations as described in example 7.

FIG. 11 shows the inhibition of cat allergic patients IgE binding to Fel d 1 by antisera obtained by immunization of rabbits with SuperCats and commercial allergen extract based vaccines.

FIG. 12 shows the inhibition of cat allergic patients IgE binding to Fel d 4 (left) and Fel d 7 (right) by antisera obtained by immunization of rabbits with SuperCats and commercial allergen extract-based vaccines.

### DESCRIPTION OF EMBODIMENTS

The peptide of the present invention derived from mature allergen Fel d 4 is a fragment of said allergen (i.e. an allergen fragment) and consists of 20 to 30 amino acid residues. “Allergen fragment”, as used herein, refers to a peptide or polypeptide stretch derived from an allergen by fragmentation.

Peptides derived from the C-terminal part of Fel d 4 after amino acid residue 120 can surprisingly induce the formation of Fel d 4 specific antibodies within a human and mammalian body and shows hypoallergenic properties. Furthermore, these peptides show no or substantially no IgE binding capacity. This latter property is important for a safe vaccine having no or substantially side effects.

The terms “of a mature allergen” and “derived from a mature allergen”, as used herein, mean that the amino acid sequences of fragments according to the present invention are obtained from the amino acid sequence of an allergen by fragmentation or truncation. Therefore, the peptide of the present invention consists of 20 to 50 consecutive amino acid residues of the mature allergen from which they are derived from. “Of a mature allergen” and “derived from a mature allergen” includes also the exchange of single amino acid residues in the aforementioned fragments. In a particularly preferred embodiment of the present invention one or more cysteine residues within amino acid positions 120 to 171 of mature allergen Fel d 4, in particular of SEQ ID No: 1, are deleted or exchanged with another amino acid residue, preferably selected from the group consisting of serine, threonine and methionine, whereby serine is most preferred. The removal or exchange of cysteine residues, for instance, is particularly advantageous to avoid the potential formation of disulphide bonds within the fragment or between two fragments.

“Mature allergen”, as defined herein, refers to the amino acid sequence of a processed allergen which lacks a signal peptide. The allergen itself is encoded by the respective gene which still contains a signal peptide. The signal peptide of an allergen can be identified by methods known in the art including sequence alignments (see e.g. Bendtsen J D et al., J Mol Biol. 340(2004):783-95; Bjorklund A K et al., Bioinformatics 21(2005):39-50). A simple method to identify the amino acid sequence of a mature allergen is to isolate the mature allergen from an allergen source and to sequence its N-terminal end. These sequence data may be compared with the sequence of the gene or the mRNA encoding the same allergen to identify the amino acid sequence of the mature allergen and of the signal peptide. Thus, the sequence of a “mature allergen” is encoded by its mRNA and/or gene and does not include cleavage products potentially obtained by post-translational modifications of primary translated mRNA or gene products apart from the potential cleavage of the signal peptide. “Mature allergen” reflects the amino acid sequence encoded by an mRNA molecule lacking the signal peptide.

Fel d 4 may have the amino acid sequence deposited under UniProt Accession number Q5VFH6:

MKLLLLCLGLILVCAHEEENVVRSNIDISKISGEWYSILLASDVK
EKIEENGSMRVFVEHIKALDNSSLSFVFHTKENGKCTEIFLVADK
TKDGVYTVVYDGYNVFSIVETVYDEYILLHLLNFDKTRPFQLVEF
YAREPDVSQKLKEKFVKYCQEHGIVNILDLTEVDRCLQARGSEVA
QDSSVE
(SEQ ID No: 2; signal peptide is shown in
italic and underlined)

Mature Fel d 4 comprises 171 amino acid residues and lacks the signal peptide as shown above in italic and underlined. The term “Fel d 4” includes also its isoforms.

According to a preferred embodiment of the present invention the peptide being derived from mature allergen Fel d 4 consists of 22 to 28, preferably 24 to 28, more preferably 25 to 27, even more preferably of 26, amino acid residues.

According to a preferred embodiment of the present invention the peptide of the present invention is derived from amino acid position 130 to 171, preferably from amino acid position 140 to 171, more preferably from amino acid position 145 to 171, more preferably from amino acid position 146 to 171, of mature Fel d 4.

According to another preferred embodiment of the present invention mature Fel d 4 comprises or consists of SEQ ID No: 1:

HEEENVVRSNIDISKISGEWYSILLASDVKEKIEENGSMRVFVK
HIKALDNSSLSFVFHTKENGKCTEIFLVADKTKDGVYTVVYDGYN
VFSIVETVYDEYILLHLLNFDKTRPFQLVEFYAREPDVSQKLKEK
FVKYCQEHGIVNILDLTEVDRCLQARGSEVAQDSSVE

According to a further preferred embodiment of the present invention the peptide consisting of 20 to 30 amino acid residues derived from amino acid position 120 to 171 of mature allergen Fel d 4 comprises or consists of amino acid sequence SEQ ID No: 3 (NILDLTEVDRCLQARGSEVAQDSSVE).

Another aspect of the present invention relates to a fusion protein or a conjugate comprising at least one peptide according to the present invention.

The peptide of the present invention consisting of 20 to 30 amino acid residues derived from amino acid position 120 to 171 of mature allergen Fel d 4 as defined above can be part of a fusion protein or a conjugate. The fusion partner or conjugation partner can be another allergen or fragment thereof or any other peptide, polypeptide or protein. The peptide of the present invention can be fused C- and/or N-terminally to a further peptidic molecule.

“Fusion protein”, as used herein, refers to a protein or polypeptide comprising the wherein the allergen fragments and/or other proteins, polypeptides or peptides are expressed and prepared as one single and unique recombinant polypeptide chain.

The term “conjugate”, as used herein, refers to a molecule formed as a result of covalent linking of at least two conjugation partners to each other. The “conjugate” of the present invention comprises at least the peptide of the present invention. Conjugation between two or more peptides, polypeptides or proteins is, for instance, achieved via an N-terminal cysteine or C-terminal cysteine amide residue added to the one of the conjugation partners resulting in a molecule containing a free sulfhydryl group. To said terminal cysteine residue any maleimide-activated polypeptide or protein may be conjugated. If the conjugation partners do not have a cysteine residue at a terminus, EDC (1-ethyl-3-(3-dimethylaminopropyl) carbodiimide hydrochloride) chemistry in order to couple amines (lysine) or carboxylic acids (glutamic, aspartic acid or 5′-phosphate) to a conjugation partner may be employed. Crosslinking between the peptide and the carrier is then for instance effected with an MBS (m-maleimidobenzoyl-N-hydroxysuccinimide ester) coupling agent.

According to a preferred embodiment of the present invention the peptide of the present invention is conjugated or fused to at least one (further) allergen fragment and/or at least one carrier protein.

At least one peptide of the present invention can be fused or conjugated to at least one allergen fragment which can be different from said peptide and/or to at least one carrier protein.

The peptide of the present invention may be coupled to a carrier protein in order to obtain a conjugation product which allows to induce the formation of allergen specific IgG antibodies more efficiently. For instance, Keyhole limpet haemocyanin (KLH) or bovine or human serum albumin can be used as carrier proteins. Also other carrier proteins like ovalbumin, thyroglobulin, tetanus toxoid or diphtheria toxoid can be used. In another embodiment of the present invention the peptide of the present invention may be fused to a carrier protein. The provision of a fusion protein comprising the peptide of the present invention and at least one carrier protein is most preferred.

“At least one allergen fragment”, as used here, refers to a further allergen fragment next to the peptide of the present invention which can also be considered as an allergen fragment. This means that that the “at least one allergen fragment” is intended to be “at least one further allergen fragment”.

According to a further preferred embodiment of the present invention the at least one allergen fragment is derived from at least one furry animal allergen, preferably from at least one cat allergen.

The at least one allergen fragment which is fused or conjugated to the peptide of the present invention may be of any allergy causing source. The at least one fragment may be derived from at least one allergen of one or more plants, insects, arthropods like mites, mammals etc, whereby mammalian allergens are particularly preferred.

According to another preferred embodiment of the present invention the at least one cat allergen is selected from the group consisting of Fel d 1 chain 1 (P30438 UniProt), Fel d 1 chain 2 (P30440 UniProt), Fel d 2 (P49064 UniProt), Fel d 3 (Q8WNR9 UniProt), Fel d 5, Fel d 6, Fel d 7 (E5D2Z5 UniProt) and Fel d 8 (F6K0R4 UniProt), preferably from the group consisting of Fel d 1 chain 1, Fel d 1 chain 2 and Fel d 7, more preferably from the group consisting of Fel d 1 chain 1, Fel d 1 chain 2, and Fel d 7.

According to a preferred embodiment of the present invention the at least one allergen fragment derived from allergens Fel d 1 chain 1, Fel d 1 chain 2, Fel d 2, Fel d 3, Fel d 5, Fel d 6, Fel d 7 and Fel d 8 comprises or consists of 20 to 40 amino acid residues.

According to another preferred embodiment of the present invention the at least one allergen fragment of the at least one cat allergen is derived from and comprises the N-terminal end or the C-terminal end of said at least one cat allergen.

It turned out that it is advantageous to use (hypoallergenic) cat allergen fragments which are derived from and comprise the N-terminal end or the C-terminal end of the allergen. If the at least one allergen fragment comprises these parts of the allergen the immune response directed to the respective cat allergen is even higher compared to fragments obtained from other parts of the cat allergens.

According to a preferred embodiment of the present invention the at least one allergen fragment derived from Fel d 1 chain 1 comprises or consists of amino acid residues 1 to 34 of mature Fel d 1 chain 1.

According to another preferred embodiment of the present invention the at least one allergen fragment derived from Fel d 1 chain 2 comprises or consists of amino acid residues 81 to 109 of mature Fel d 1 chain 2.

According to a further preferred embodiment of the present invention the at least one allergen fragment derived from Fel d 7 comprises or consists of amino acid residues 61 to 97 and/or amino acid residues 124 to 162 of mature Fel d 7.

According to a preferred embodiment of the present invention the carrier protein is a viral protein or a fragment thereof consisting of 50 to 300, preferably 60 to 250, more preferably 80 to 200, more preferably 100 to 200, amino acid residues.

According to another preferred embodiment of the present invention the viral protein is a capsid protein.

According to a further preferred embodiment of the present invention the viral protein is derived from a virus of the hepadnaviridae family.

According to a preferred embodiment of the present invention the virus of the hepadnaviridae family is a Hepatitis B virus.

According to a preferred embodiment of the present invention the viral protein of the Hepatitis B virus is PreS, PreS1 or PreS2.

A fragment of a hepatitis B PreS polypeptide consists preferably of at least 30, preferably at least 40, more preferably at least 50, consecutive amino acid residues and may comprise PreS1 and/or PreS2 of the hepatitis B PreS polypeptide.

The Hepatitis B PreS polypeptide in its function as a carrier protein and being used as a fusion or conjugation partner for the peptide of the present invention may comprise or consist of the following amino acid sequence (SEQ ID No: 4):

GGWSSKPRKGMGTNLSVPNPLGFFPDHQLDPAFGANSNNPDWDFN
PIKDHWPAANQVGVGAFGPGLTPPHGGILGWSPQAQGILTTVSTI
PPPASTNRQSGRQPTPISPPLRDSHPQAMQWNSTAFHQALQDPRV
RGLYFPAGGSSSGTVNPAPNIASHISSISARTGDPVTN

The Hepatitis B PreS polypeptide may consist of an amino acid sequence which is at least 70%, preferably at least 80%, more preferably at least 90%, more preferably at least 95%, more preferably at least 99%, in particular 100%, identical to SEQ ID No: 4.

According to a preferred embodiment of the present invention the fusion protein comprises at least two peptides consisting of 20 to 30 amino acid residues derived from amino acid position 120 to the C-terminal end of mature allergen Fel d 4 according to the present invention and at least two allergen fragments as defined above.

The combination of allergen fragments is particularly preferred since it allows the creation of a fusion protein which is able to induce the formation of antibodies directed to more cat allergens, for instance. If more than one peptide and allergen fragment of the same source are combined in one single fusion protein the order these peptides and allergen fragments does not correspond to the order in the naturally occurring allergen.

According to a further preferred embodiment of the present invention two to eight, preferably two to six, of the at least one peptide and two to eight, preferably two to six, of at least one allergen fragment are fused to the N-terminal end and the C-terminal end of the at least one carrier protein.

The peptides and allergen fragments of the present invention can be fused to the N- and/or C-terminal end of a carrier protein. It is particularly preferred that the peptides and allergen fragments are fused to N- and C-terminal ends of the carrier protein since it turned out that this results in an even higher induction of allergen specific antibodies within a human and mammal.

According to a preferred embodiment of the present invention two peptides of the at least one peptide are located adjacent to each other within the fusion protein.

According to another preferred embodiment of the present invention the fusion protein comprises two peptides comprising or consisting of amino acid residues 146 to 171 of mature Fel d 4, two allergen fragments comprising or consisting of amino acid residues 1 to 34 of mature Fel d 1 chain 1, two allergen fragments comprising or consisting of amino acid residues 81 to 109 of mature Fel d 1 chain 2, two allergen fragments comprising or consisting of amino acid residues 81 to 109 of mature Fel d 1 chain 2, two allergen fragments comprising or consisting of amino acid residues 61 to 97 of mature Fel d 7 and two allergen fragments comprising or consisting of amino acid residues 124 to 162 of mature Fel d 7.

According to a preferred embodiment of the present invention said fusion protein comprises or consists of amino acid sequence SEQ ID No: 5, SEQ ID No: 6, SEQ ID No: 7, SEQ ID No: 8, SEQ ID No: 9, SEQ ID No: 10, SEQ ID No: 11, SEQ ID No: 12, SEQ ID No: 13 or SEQ ID No: 14, preferably SEQ ID No: 5, SEQ ID No: 7, SEQ ID No: 9, SEQ ID No: 11 or SEQ ID No: 13.

It turned surprisingly out that these fusions proteins are able to induce the formation of cat allergen specific antibodies in humans and mammals so that most widely spread allergens are covered. SEQ ID No: 5, SEQ ID No: 9 and SEQ ID No: 13 are particularly preferred fusion proteins.

A further aspect of the present invention relates to a peptide or a fusion protein or a conjugate according to the present invention for the use in the prevention or treatment of cat allergy.

Another aspect of the present invention relates to a peptide or a fusion protein or a conjugate according to the present invention for the use in the prevention or treatment of a furry animal allergy, preferably a dog or horse allergy.

The fusions proteins and conjugates of the present invention induce the formation of antibodies which are also able to bind allergens from another source than cat. Hence, the fusion proteins and conjugates of the present invention can be used in the prevention or treatment of another furry animal allergy. Particularly preferred are dog and horse allergies.

A further aspect of the present invention relates to a nucleic acid molecule encoding a peptide or a fusion protein according to the present invention.

The nucleic acid molecule of the present invention can be a RNA or DNA molecules.

Another aspect of the present invention relates to a vector comprising a nucleic acid molecule according to the present invention.

The vector of the present invention can be an expression or a cloning vector. The vector can be a bacterial, fungal, insect, viral or mammalian vector.

The vector of the present invention may preferably be employed for cloning and expression purposes in various hosts like bacteria, yeasts, filamentous fungi, mammalian cells, insect cells, plant cells or any other prokaryotic or eukaryotic cells. Therefore, said vector comprises besides a nucleic acid encoding for a fusion protein according to the present invention host specific regulatory sequences.

An aspect of the present invention relates to a host cell comprising a nucleic acid molecule or a vector according to the present invention.

A further aspect of the present invention relates to a vaccine formulation comprising a peptide, a fusion protein, a nucleic acid molecule and/or a vector according to the present invention.

According to a preferred embodiment of the present invention the vaccine formulation of the present invention can be used in the prevention or treatment of a furry animal allergy, preferably a cat, dog and/or horse allergy.

According to another preferred embodiment of the present invention said formulation comprises 10 ng to 1 g, preferably 100 ng to 10 mg, especially 0.5 μg to 200 μg of said fusion protein, nucleic acid molecule or vector.

According to a particularly preferred embodiment of the present invention the fusion protein, nucleic acid molecule or vector of the present invention is administered to an individual at least once in an amount of 0.01 pg/kg body weight to 5 mg/kg body weight, preferably 0.1 pg/kg body weight to 2 mg/kg body weight.

According to further preferred embodiment of the present invention the fusion protein, nucleic acid molecule or vector is administered to a patient in an amount of 5 to 100 μg, preferably 10 to 80 μg, either independent from the body weight (i.e. a dose may comprise 15, 20, 25, 30, or 80 μg) or per kg body weight.

The amount of fusion protein, nucleic acid molecule or vector that may be combined with excipients to produce a single dosage form will vary depending upon the host treated and the particular mode of administration. The dose of the fusion protein, nucleic acid molecule or vector may vary according to factors such as the disease state, age, sex and weight of the individual, and the ability to elicit the desired antibody response in the individual. The dosage regime may be adjusted to provide the optimum therapeutic response. The dose of the fusion protein, nucleic acid molecule or vector may also be varied to provide optimum preventative dose response depending upon the circumstances. For instance, the fusion protein, nucleic acid molecule or vector of the present invention may be administered to an individual at intervals of several days, one or two weeks or even months depending always on the level of allergen specific IgG induction.

In a preferred embodiment of the present invention the fusion protein, nucleic acid molecule or vector of the present invention are applied between 2 and 10, preferably between 2 and 7, even more preferably up to 5 times, whereby the time span between the dose applications is between 2 to 60 days, preferably 5 to 40 days, more preferably 14 to 28 days. In a further preferred embodiment of the present invention, single booster applications are given between 3 months and 5 years following the first dosing schedule. These booster application may be repeated between 2 and 10 times, preferably between 2 and 5 times and most preferably between 2 and 3 times, to keep the IgG levels high. In a particularly preferred embodiment the time interval between the subsequent vaccinations is chosen to be between 2 weeks and 5 years, preferably between 3 weeks and up to 3 years, more preferably between 3 weeks and 1 year. The repeated administration of the fusion protein, nucleic acid molecule or vector of the present invention may maximize the final effect of the treatment.

In a particularly preferred embodiment of the present invention the fusion protein, nucleic acid molecule or vector of the present invention may be applied using 3 to 6, preferably 5, monthly injections followed by booster injections as mentioned above given every 1 to 6, preferably 3 to 4 months, for at least one, preferably at least two, more preferably from two to six, more preferably from three to five years.

According to another preferred embodiment of the present invention the vaccine formulation further comprises at least one adjuvant, pharmaceutical acceptable excipient and/or preservative.

The fusion protein, nucleic acid molecule, vector and pharmaceutical preparation of the present invention can be administrated subcutaneously, intramuscularly, mucosally etc. Depending on the dosage form and administration route the fusion protein, nucleic acid molecule or vector of the present invention may be combined with excipients, diluents, adjuvants and/or carriers. A preferred adjuvant is aluminum hydroxide. Suitable protocols for the production of vaccine formulations are known to the person skilled in the art and can be found e.g. in “Vaccine Protocols” (A. Robinson, M. P. Cranage, M. Hudson; Humana Press Inc., U. S.; 2nd edition 2003).

The fusion protein of the present invention may be formulated also with other adjuvants regularly used in vaccines. For instance, suitable adjuvants may be MF59, aluminium phosphate, calcium phosphate, cytokines (e.g. IL2, IL-12, GM-CSF), saponins (e.g. QS21), MDP derivatives, CpG oligonucleotides, LPS, MPL, polyphosphazenes, emulsions (e.g. Freund's, SAF), liposomes, virosomes, iscoms, cochleates, PLG microparticles, poloxamer particles, virus-like particles, heat-labile enterotoxin (LT), cholera toxin (CT), mutant toxins (e.g. LTK63 and LTR72), microparticles and/or polymerized liposomes. Suitable adjuvants are commercially available as, for example, ASO1B (MPL and QS21 in a liposome formulation), ASO2A, AS15, AS-2, AS-03 and derivatives thereof (GlaxoSmithKline, USA); CWS (cell-wall skeleton), TDM (trehalose-6,6′-dimycolate), LeIF (Leishmania elongation initiation factor), aluminium salts such as aluminium hydroxide gel (alum) or aluminium phosphate; salts of calcium, iron or zinc; an insoluble suspension of acylated tyrosine; acylated sugars; catatonically or anionically derivatized polysaccharides; polyphosphazenes; biodegradable microspheres; monophosphoryl lipid A and quil A. Cytokines, such as GM-CSF or interleu-kin-2, -7 or -12 may also be used as adjuvants. Preferred adjuvants for use in eliciting a predominantly Thl-type response include, for example, a combination of monophos-phoryl lipid A, preferably 3-O-deacylated monophosphoryl lipid A (3D-MPL), optionally with an aluminum salt. Aqueous formulations comprising monophosphoryl lipid A and a surfactant have been described in WO 98/43670.

Another preferred adjuvant is a saponin or saponin mimetic] or derivatives, preferably QS21 (Aquila Biophar-maceuticals Inc.), which may be used alone or in combination with other adjuvants. For example, an enhanced system involves the combination of a monophosphoryl lipid A and saponin derivative, such as the combination of QS21 and 3D-MPL. Other preferred formulations comprise an oil-in-water emulsion and tocopherol. A particularly potent adjuvant formulation is QS21, 3D-MPL and tocopherol in an oil-in-water emulsion. Additional saponin adjuvants for use in the present invention include QS7 (described in WO 96/33739 and WO 96/11711) and QS17 (described in U.S. Pat. No. 5,057,540 and EP 0 362 279 B1).

The vaccine formulation comprising the fusion protein of the present invention comprises most preferably aluminium hydroxide as adjuvant.

### EXAMPLES

### Example 1: Identification of the Cat Allergens Recognized by IgE Antibodies of the Majority of Cat Allergic Patients with High Allergenic Activity

In order to identify the most relevant cat allergens to be included in a cat allergy vaccine two parameters were evaluated. The first parameter was the frequency of IgE recognition of the cat allergens by cat allergic patients and the second was the allergenic activity which corresponds to the potency of a given allergen molecule to induce allergic inflammation in a sensitized patient.

The frequency of IgE reactivity was evaluated by testing cat allergens Fel d 1 (Fel d 1.A.0101(chain 1), Fel d 1.B.0101 (chain 1)), Fel d 2 (Fel d 2.0101), Fel d 3 (Fel d 3.0101), Fel d 4 (Fel d 4.0101), Fel d 6 (Fel d 6.0101), Fel d 7 (Fel d 7.0101]) and Fel d 8 (Fel d 8.0101) for IgE reactivity in cat allergy patients using the ImmunoCAP technology (van Hage M et al. J Allergy Clin Immunol. 140(2017):974-977). The aforementioned cat allergens were biotinylated, bound to streptavidin CAPs and used for IgE reactivity testing by ImmunoCAP in two cohorts of cat allergic patients, one from Sweden (n=72) and one from Russia (n=73). The allergenic activity of the cat allergens was performed by basophil activation testing using rat basophil leukemia cells which express the human high affinity receptor for IgE and hence can be loaded serum IgE from cat allergic patients.

The basophil activation is induced by addition of allergens which crosslink IgE and leads to of betahexosaminidase which can be measured in the cell culture supernatants as described in Rodriguez-Dominguez A et al. (J Allergy Clin Immunol. 146(2020):1097-1108).

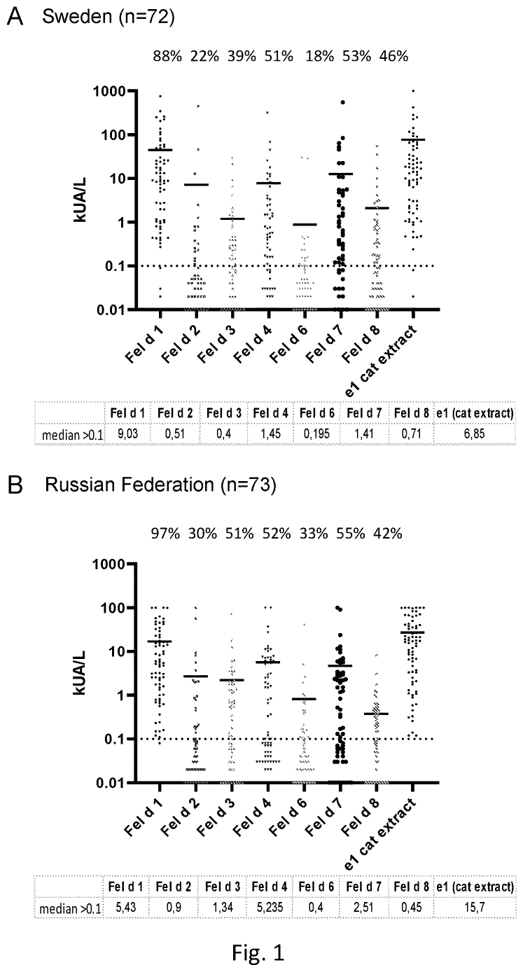

Prevalences of allergen-specific IgE recognition in Swedish vs Russian patients were as follows: Fel d 1, 88% vs 97%; Fel d 2, 22% vs 30%; Fel d 3; 39% vs 51%; Fel d 4, 51% vs 52%; Fel d 6; 18% vs 33%; Fel d 7, 53% vs 55% and Fel d 8, 46% vs 42% (see FIG. 1). Thus cat allergens which were most frequently recognized by patients IgE were Fel d 1, Fel d 7, Fel d 4 and Fel d 3 (see FIG. 1).

Specific IgE levels (in kUA/L, y-axis) to cat allergens and cat extract (x-axis), determined for two populations of patients (A, Swedish; B, Russian) with reactivity to cat extract are displayed as scatter plots (FIG. 1). Horizontal lines represent the cut-off values of 0.1 kUA/l. Medians IgE levels were calculated only for values>0.1 kUA/L. Percentages of IgE-positive sera for the individual allergens are displayed above the plots. IgE equal or above 100 kUA/L was set at 100 for the Russian population.

The allergenic activity of cat allergens was determined using basophil activation assays. RBL (Rat Basophilic Leukemia) cells were loaded with sera from 17 Swedish cat allergic patients and from a non-allergic person and then stimulated with decreasing allergen concentrations for each allergen. According to the basophil activation tests, Fel d 1, Fel d 4 and Fel d 7 were identified as the most frequently and most allergenic cat allergen molecules to include in a cat allergy vaccine whereas the other cat allergen molecules were not frequently recognized and/or exhibited low allergenic activity.

### Example 2: Fel d4-Derived Peptide P9 and Fel d 7-Derived Peptides P3 and P5 are Part of the Major IgE Epitope-Containing Areas of Fel d4 and Fel d 7, Respectively

In order to investigate the IgE epitopes on Fel d 4 and Fel d 7, several fragments of these allergens have been chemically synthesized.

TABLE 1
The amino acid sequence of Fel d 4 and Fel d 7 derived
peptides (cysteine residues may be added to the N- or
C-terminus to facilitate coupling reactions)

| | | | | SEQ ID |
|---|---|---|---|---|
| Allergen | Peptide | Amino acid sequence | Position | No: |
| Fel d 4 | P1 | EEENVVRSNIDISKISGEWYSILLASDVKE | 1-35 | 15 |
| | | KIEE | | |
| | P3 | HIKALDNSSLSFVFHTKENGKCTEIFLVAD | 45-87 | 16 |
| | | KTKDGVYTVVYDG | | |
| | P6 | HLLNFDKTRPFQLVEFYAREPDVSQKLKE | 105-139 | 17 |
| | | KFVKYC | | |
| | P9 | NILDLTEVDRCLQARGSEVAQDSSVE | 146-171 | 18 |
| Fel d 7 | P1 | QDPPASGEDTMAMSGKWYLKAMITDRE | 1-37 | 19 |
| | | TSWKKPELVT | | |
| | P2 | KKPELVTPMTLTVLEGGNLKAETTLLING | 31-65 | 20 |
| | | QCKEVE | | |
| | P3 | CKEVELILEKTSEPKKYTTYGGKRVVYIE | 61-97 | 21 |
| | | PTEVKDHY | | |
| | P4 | CEGEMQGEQARMAKLVGRDPESNEEALE | 101-135 | 22 |
| | | NFREFLR | | |
| | P5 | EEALENFREFLRAKGFNQEIFSPKQSDTCP | 124-162 | 23 |
| | | PGTDQEPEV | | |

Then rabbits were immunized with KLH-coupled peptides and for comparison with the complete allergens (i.e., rFel d 4, rFel d 7) with three injections, first booster after 4 weeks and second booster after 3 weeks. Then competitive ELISAs with anti-peptides specific antisera were performed to determine the inhibitory effect of peptide specific IgGs on allergic patients IgE binding to the allergens as described (Curin M et al. Front Immunol.12(2021):687294).

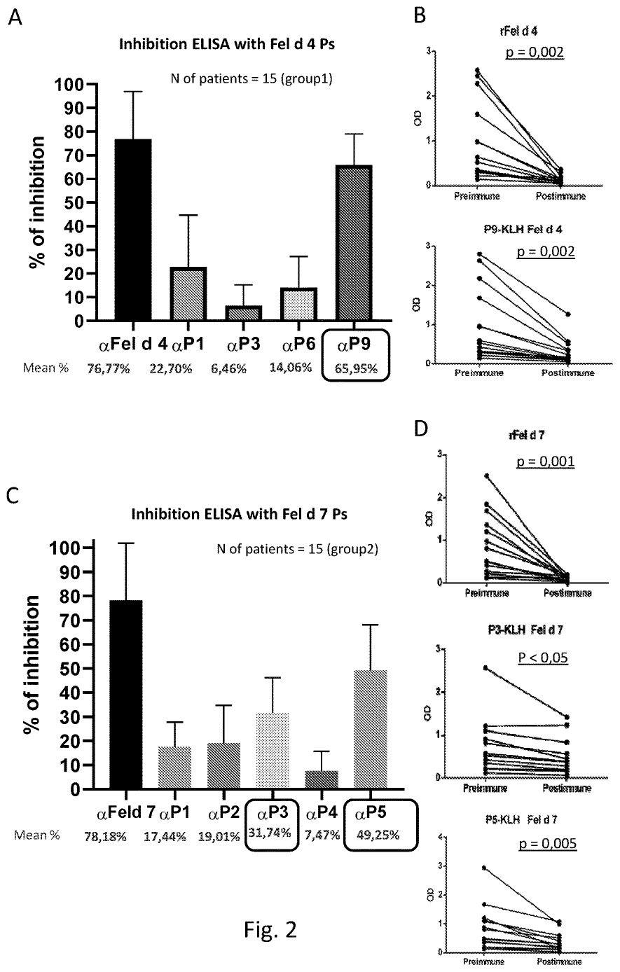

The average percentage of inhibition of antisera against Fel d 4-derived peptides was lowest for P3—6.46%. Some inhibition was observed for antisera to P1 and P6; 22.70% and 14.06%, respectively. The best inhibition was obtained for antibodies specific for P9 (i.e., 65.95%) which was comparable for the inhibition obtained with the antiserum against complete-Fel d 4 (FIG. 2). The histograms in FIG. 2A and FIG. 2C show the mean percentage of inhibition+/−SD for patients grouped by results for antisera against peptides and complete allergens. The graphs in FIG. 2B and FIG. 2D show the binding of allergic patients IgE (n=15) (optical density—OD values) after pre-incubation of plate-bound allergens with pre-immune sera or antisera raised against peptides or complete allergens (x-axes). (N of patients=15). The dots indicate the OD values, pair values are connected by lines. Differences between groups were compared by Student's dependent t-test for paired samples. P-values<0.05 were considered significant.

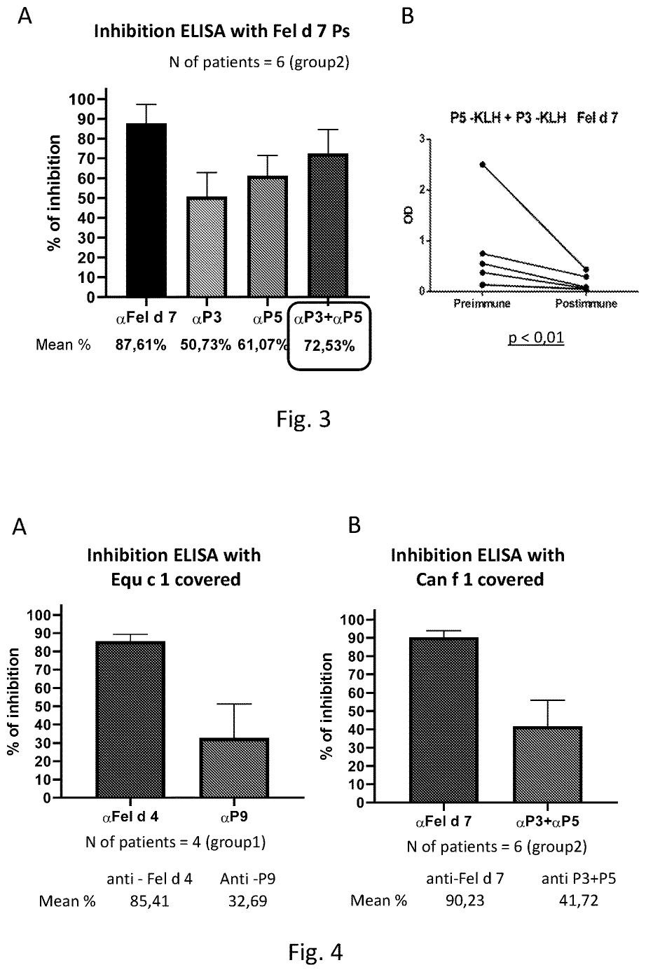

The average percentage of inhibition for antisera raised against Fel d 7 peptides was lowest for P4—7.47%. Some inhibition was observed for antisera raised against P1 and P2; 17.44% and 19.01%, respectively. When inhibition of patients IgE binding was performed with a combination of antisera specific for Fel d 7-derived peptides P3 and P5 was done the percentage of mean inhibition was 72.53% which was similar to that achieved with antibodies raised against complete Fel d 7 (i.e., 87.61%) (FIG. 3). The histogram of FIG. 3A shows the percentage of inhibition for patients grouped by results for peptides and mix of P3 with P5 Fel d 7. The highest percentage of inhibition for all patients is observed for mix of peptides. The graph of FIG. 3B shows the associated OD values for ELISA results with pre-immune serum and immune (N of patients=6). The dots indicate the OD values, pair values are connected by a line. Differences between groups were compared by Student's dependent t-test for paired samples. P-values<0.05 were considered significant.

Antisera raised against Fel d 7-derived P3 and P5 also inhibited IgE binding of allergic patients (n=6) to the cross-reactive allergen Can f 1 (FIG. 4A). Likewise, antibodies against the Fel d 4-derived peptide P9 inhibited IgE binding of allergic patients (n=4) to the Fel d 4 homologous allergen Equ c 1 from horse (FIG. 4B). The histograms show the percentage of inhibition by competitive ELISA with cross-reactive allergens Can f 6 (A) and Equ c 1 (B) by anti-peptide antisera.

These results identify the C-terminal portion of Fel d 4 as a major IgE epitope. Furthermore, Fel d 7-derived peptides P3 and P5 are derived from major IgE-reactive areas of Fel d 7 (Table 1). These newly identified peptides can therefore be used for the construction of a cat vaccine inducing protective IgG antibodies against Fel d 4 and Fel d 7.

### Example 3: Recombinant PreS Fusion Proteins Containing Hypoallergenic Peptides from Feld 1, Fel d 4 and Fel d 7

The construction, expression and purification of fusion proteins comprising hepatitis B virus-derived PreS and hypoallergenic peptides of the major cat allergen Fel d 1 for allergen-specific immunotherapy of cat allergy has been previously described (Niespodziana K et al. J Allergy Clin Immunol. 127(2011):1562-70). In the aforementioned study a fusion protein comprising PreS and 2 copies of peptide 1 from Fel d 1, designated PreS-2×P1, a fusion protein comprising PreS and two copies of Fel d 1-derived peptide 5, designated PreS-2×P5, and a fusion protein comprising PreS one copy of peptide 1 and one copy of peptide 5, designated PreS-P1-P5, have been used. The three recombinant fusion proteins showed reduced IgE reactivity and allergenic activity when tested in cat allergic patients and PreS-2×P1 and PreS-P1-P5 were found to be superior to induce upon immunization of mice and rabbits IgG antibodies that blocked allergic patients IgE binding to Fel d 1 as compared to PreS-2×P5. Since both Fel d 1-derived peptides P1 and P5 appeared to be important for the induction of IgG antibodies blocking cat allergic patients IgE binding to Fel d 1, both peptides were used for the construction of a combination vaccine for Fel d 1, Fel d 4 and Fel d 7.

TABLE 2
The amino acid sequence of Fel d 1, Fel d 4
and Fel d 7 derived peptides used in the
fusion proteins according to Table 3

| | | | SEQ |
|---|---|---|---|
| Peptide | Amino acid sequence | Position | ID No: |
| P1 (Fel d 1) | EICPAVKRDVDLFLTGTPD | 1-34 | 24 |
| | EYVEQVAQYKALPVV | (chain 1) | |
| P5 (Fel d 1) | MTTISSSKDCMGEAVQNTV | 81-109 | 25 |
| | EDLKLNTLGR | (chain 2) | |
| P9 (Fel d 4) | NILDLTEVDRCLQARGSEV | 146-171 | 18 |
| | AQDSSVE | | |
| P3 (Fel d 7) | CKEVELILEKTSEPKKYTT | 61-97 | 21 |
| | YGGKRVVYIEPTEVKDHY | | |
| P5 (Fel d 7) | EEALENFREFLRAKGFNQE | 124-162 | 23 |
| | IFSPKQSDTCPPGTDQEPEV | | |

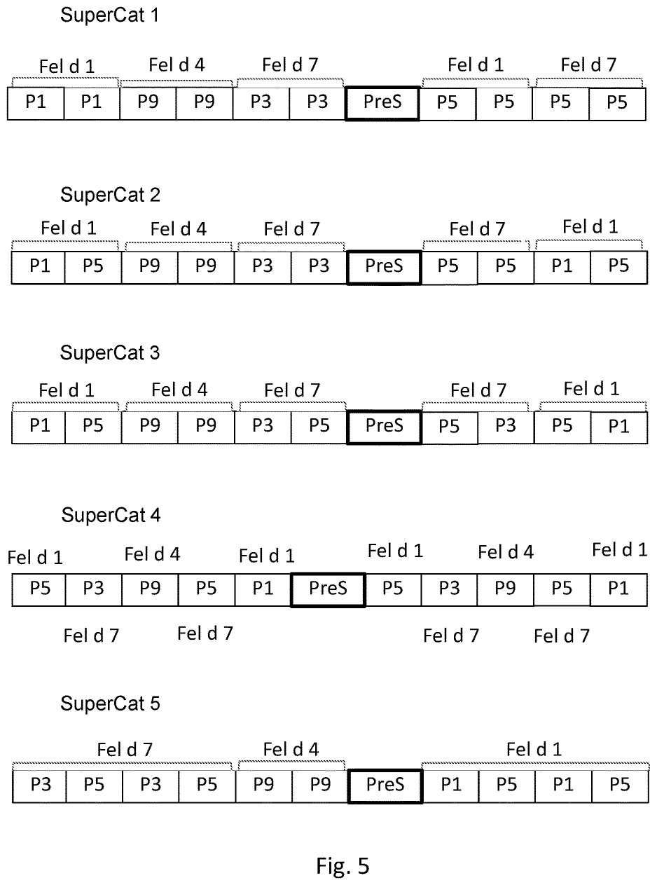

Five fusion proteins containing two copies of the Fel d 1-derived peptides P1 and P5 together with two copies of the Fel d 4-derived peptide P9 and two copies of each of the two Fel d 7-derived peptides P3 and P9 have been designed (FIG. 5). The five fusion proteins designated SuperCat 1 to SuperCat 5 contained the identical allergen-derived peptides but in different order.

TABLE 3
Amino acid sequences of the fusion proteins (His-tag (italic and bold)
added to the N-terminal end to facilitate purification)

| | | SEQ ID |
|---|---|---|
| Name | Amino acid sequence | No: |
| SuperCat 1 | EICPAVKRDVDLFLTGTPDEYVEQVAQYKALPVVEICPAV | 5 (without |
| | KRDVDLFLTGTPDEYVEQVAQYKALPVVNILDLTEVDRCL | His-tag) |
| | QARGSEVAQDSSVENILDLTEVDRCLQARGSEVAQDSSVE | 6 (with |
| | KEVELILEKTSEPKKYTTYGGKRVVYIEPTEVKDHYKEVEL | His-tag) |
| | ILEKTSEPKKYTTYGGKRVVYIEPTEVKDHYGGWSSKPRK | |
| | GMGTNLSVPNPLGFFPDHQLDPAFGANSNNPDWDENPIKD | |
| | HWPAANQVGVGAFGPGLTPPHGGILGWSPQAQGILTTVST | |
| | IPPPASTNRQSGRQPTPISPPLRDSHPQAMQWNSTAFHQAL | |
| | QDPRVRGLYFPAGGSSSGTVNPAPNIASHISSISARTGDPVT | |
| | NMTTISSSKDCMGEAVQNTVEDLKLNTLGRMTTISSSKDC | |
| | MGEAVQNTVEDLKLNTLGREEALENFREFLRAKGFNQEIF | |
| | SPKQSDTCPPGTDQEPEVEEALENFREFLRAKGFNQEIFSPK | |
| | QSDTCPPGTDQEPEVHHHHHH | |
| SuperCat 2 | EICPAVKRDVDLFLTGTPDEYVEQVAQYKALPVVMTTISSS | 7 (without |
| | KDCMGEAVQNTVEDLKLNTLGRNILDLTEVDRCLQARGS | His-tag) |
| | EVAQDSSVENILDLTEVDRCLQARGSEVAQDSSVEKEVELI | 8 (with |
| | LEKTSEPKKYTTYGGKRVVYIEPTEVKDHYKEVELILEKTS | His-tag) |
| | EPKKYTTYGGKRVVYIEPTEVKDHYGGWSSKPRKGMGTN | |
| | LSVPNPLGFFPDHQLDPAFGANSNNPDWDFNPIKDHWPAA | |
| | NQVGVGAFGPGLTPPHGGILGWSPQAQGILTTVSTIPPPAST | |
| | NRQSGRQPTPISPPLRDSHPQAMQWNSTAFHQALQDPRVR | |
| | GLYFPAGGSSSGTVNPAPNIASHISSISARTGDPVTNEEALE | |
| | NFREFLRAKGFNQEIFSPKQSDTCPPGTDQEPEVEEALENFR | |
| | EFLRAKGFNQEIFSPKQSDTCPPGTDQEPEVEICPAVKRDV | |
| | DLFLTGTPDEYVEQVAQYKALPVVMTTISSSKDCMGEAVQ | |
| | NTVEDLKLNTLGRHHHHHH | |
| SuperCat 3 | EICPAVKRDVDLFLTGTPDEYVEQVAQYKALPVVMTTISSS | 9 (without |
| | KDCMGEAVQNTVEDLKLNTLGRNILDLTEVDRCLQARGS | His-tag) |
| | EVAQDSSVENILDLTEVDRCLQARGSEVAQDSSVEKEVELI | 10 (with |
| | LEKTSEPKKYTTYGGKRVVYIEPTEVKDHYEEALENFREFL | His-tag) |
| | RAKGFNQEIFSPKQSDTCPPGTDQEPEVGGWSSKPRKGMG | |
| | TNLSVPNPLGFFPDHQLDPAFGANSNNPDWDFNPIKDHWP | |
| | AANQVGVGAFGPGLTPPHGGILGWSPQAQGILTTVSTIPPP | |
| | ASTNRQSGRQPTPISPPLRDSHPQAMQWNSTAFHQALQDP | |
| | RVRGLYFPAGGSSSGTVNPAPNIASHISSISARTGDPVTNEE | |
| | ALENFREFLRAKGFNQEIFSPKQSDTCPPGTDQEPEVKEVE | |
| | LILEKTSEPKKYTTYGGKRVVYIEPTEVKDHYMTTISSSKD | |
| | CMGEAVQNTVEDLKLNTLGREICPAVKRDVDLFLTGTPDE | |
| | YVEQVAQYKALPVVHHHHHH | |
| SuperCat 4 | MTTISSSKDCMGEAVQNTVEDLKLNTLGRKEVELILEKTSE | 11 |
| | PKKYTTYGGKRVVYIEPTEVKDHYNILDLTEVDRCLQARG | (without |
| | SEVAQDSSVEEEALENFREFLRAKGFNQEIFSPKQSDTCPPG | His-tag) |
| | TDQEPEVEICPAVKRDVDLFLTGTPDEYVEQVAQYKALPV | 12 (with |
| | VGGWSSKPRKGMGTNLSVPNPLGFFPDHQLDPAFGANSN | His-tag) |
| | NPDWDFNPIKDHWPAANQVGVGAFGPGLTPPHGGILGWS | |
| | PQAQGILTTVSTIPPPASTNRQSGRQPTPISPPLRDSHPQAM | |
| | QWNSTAFHQALQDPRVRGLYFPAGGSSSGTVNPAPNIASHI | |
| | SSISARTGDPVTNMTTISSSKDCMGEAVQNTVEDLKLNTLG | |
| | RKEVELILEKTSEPKKYTTYGGKRVVYIEPTEVKDHYNILD | |
| | LTEVDRCLQARGSEVAQDSSVEEEALENFREFLRAKGFNQ | |
| | EIFSPKQSDTCPPGTDQEPEVEICPAVKRDVDLFLTGTPDEY | |
| | VEQVAQYKALPVVHHHHHH | |
| SuperCat 5 | KEVELILEKTSEPKKYTTYGGKRVVYIEPTEVKDHYEEALE | 13 |
| | NFREFLRAKGFNQEIFSPKQSDTCPPGTDQEPEVKEVELILE | (without |
| | KTSEPKKYTTYGGKRVVYIEPTEVKDHYEEALENFREFLR | His-tag) |
| | AKGFNQEIFSPKQSDTCPPGTDQEPEVNILDLTEVDRCLQA | 14 (with |
| | RGSEVAQDSSVENILDLTEVDRCLQARGSEVAQDSSVEGG | His-tag) |
| | WSSKPRKGMGTNLSVPNPLGFFPDHQLDPAFGANSNNPD | |
| | WDFNPIKDHWPAANQVGVGAFGPGLTPPHGGILGWSPQA | |
| | QGILTTVSTIPPPASTNRQSGRQPTPISPPLRDSHPQAMQWN | |
| | STAFHQALQDPRVRGLYFPAGGSSSGTVNPAPNIASHISSIS | |
| | ARTGDPVTNEICPAVKRDVDLFLTGTPDEYVEQVAQYKAL | |
| | PVVMTTISSSKDCMGEAVQNTVEDLKLNTLGREICPAVKR | |
| | DVDLFLTGTPDEYVEQVAQYKALPVVMTTISSSKDCMGEA | |
| | VQNTVEDLKLNTLGRHHHHHH | |

Recombinant SuperCat 1 to SuperCat 5 PreS-fusion proteins were expressed with a C-terminal hexahistidine tag to facilitate purification via nickel-affinity chromatography as described (Niespodziana K et al. J Allergy Clin Immunol. 127(2011):1562-70) and purified as follows: His-tagged PreS fusion proteins were purified under denaturing conditions. To destroy the bacterial cell walls, pellets were treated by freeze and thaw method (3 cycles), then re-suspended in 30 ml Lysis Buffer B (8M Urea, 100 mM NaH₂PO₄, 10 mM Tris-Cl, pH 8). After centrifugation the pellet was discarded and the supernatant was incubated with 2 ml of Ni-agarose (Quiagen) for 2 h at room temperature on the shaker. The mixture was then poured into propylene tubes and impurities were eluted with 10 ml of Wash Buffer C (8M Urea, 100 mM NaH₂PO₄, 10 mM Tris-Cl pH 6.3). To eluate the SuperCat1-5 from the Ni-agarose two variants of Elution Buffer with decreasing pH were used: Elutions 1-3 by Buffer D (8M Urea, 100 mM NaH₂PO₄, 10 mM Tris-Cl pH 5.9), Elutions 4-7 by Buffer E (8M Urea, 100 mM NaH₂PO₄, 10 mM Tris-Cl, pH 4.5). Urea was removed by means of dialysis against the step-gradual decrease in the molar Urea concentration from 8M to 1M Urea in PBS (pH 7.4), and then, against PBS (pH 7.4) 3 times at least for 8 h.

### Example 4: IgE Reactivity and Allergenic Activity of SuperCats 1 to 5 as Compared to a Mix of Fel d 1, Fel d 4 and Fel d 7 is Reduced to a Similar Extent

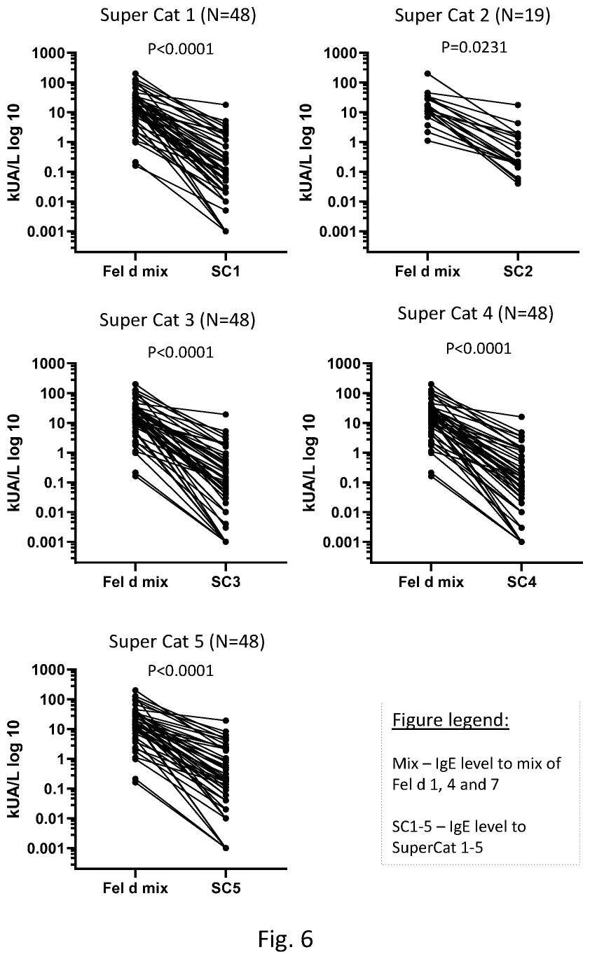

The IgE reactivity of recombinant SuperCat 1 to 5 proteins was compared with an equimolar mix of Fel d 1, Fel d 4 and Fel d 7 by ImmunoCap testing of sera from cat allergic patients (N=19 and N=29 from an Austrian and a Russian cohort, respectively) and, for control purposes, with sera from 2 non-allergic subjects. Streptavidin ImmunoCAPs (0212 ImmunoCAP, Thermo Fisher Scientific/Phadia) were used to prepare ImmunoCAPs containing the Fel d 1, 4, 7 allergens and the SuperCat fusion proteins as described (Huang H J et al. J Allergy Clin Immunol. 142(2018):1656-1659). The measurement of specific IgE levels was performed according to the manufacturer's instructions on an ImmunoCAP 100 instrument (Thermo Fisher Scientific/Phadia). The IgE reactivity of SuperCats 1 to 5 was significantly reduced compared to the allergen mix and this reduction was comparable for each of the Supercar proteins (FIG. 6). Significant reduction of IgE reactivity of SuperCat antigens as compared to the mix of Fel d 1, Fel d 4, and Fel d 7 allergens. IgE levels are expressed as kUA/L on a logarithmic scale (y-axes). The dots indicate the IgE levels, paired values (i.e., when patients were tested with the allergen mix and SuperCat antigens) are connected by a line. Differences between groups were compared by Student's dependent t-test for paired samples. P-values<0.05 were considered significant (abbreviations FIG. 6: mix—mix of Fel d 1, 4 and 7; SC1-5—SuperCats 1 to 5).

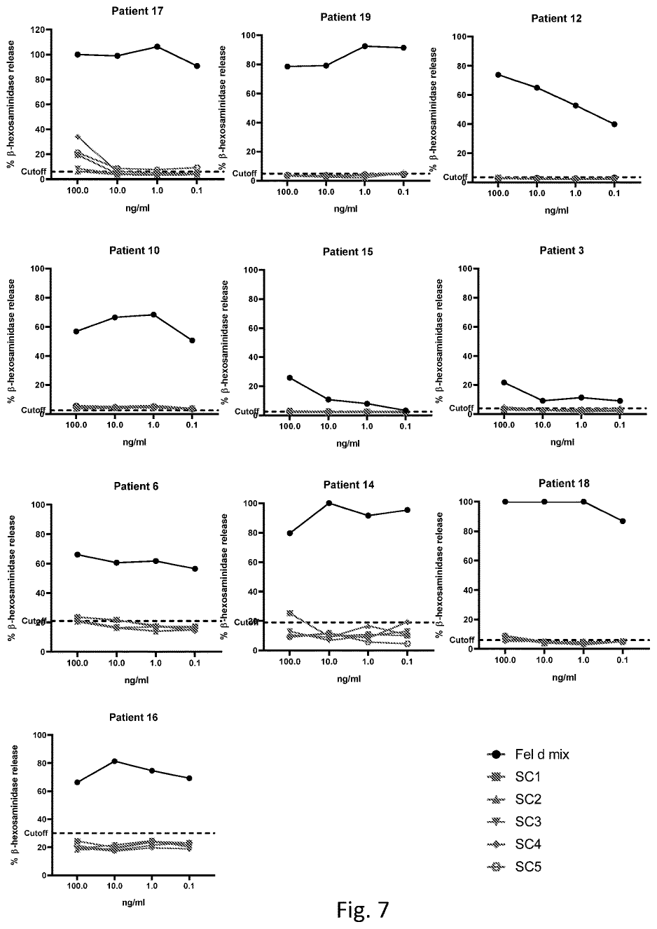

Next, the allergenic activity of the SuperCat 1 to 5 proteins in basophil activation tests using RBL cells expressing the human high-affinity IgE receptor FcϵRI in cat allergic subjects. We performed the RBL assay using a series of dilutions of antigens (100 ng/ml, 10 ng/ml, 1 ng/ml, 0.1 ng/ml of a mix of Fel d 1, 4, and 7, or equimolar concentrations of SuperCats 1-5) and obtained the following results: The mix of cat allergens induced a release of β-hexosaminidase from basophils already at very low concentrations (i.e., 0.1 ng/ml) in most of the tested patients, whereas SuperCats 1-5 did not cause degranulation of basophils in most of patients (FIG. 7). RBL cells were loaded with sera from cat allergic patients (numbers 3, 6, 10, 12, 14, 15, 16, 17, 18 and 19) and then stimulated with decreasing antigen concentrations (x-axes; Fel d mix (Fel d 1+Fel d 7+Fel d 4), SC1-5: SuperCat1, Supercat2, SuperCat3, SuperCat4, SuperCat5). β-hexosaminidase releases are expressed as percentages of total mediator contents of cells after detergent lysis (y-axes). The horizontal cut-off line corresponds to % of release of basophils after medium exposure (negative control). Abbreviations: SC1-5—SuperCats 1-5. Only in one patient with extremely high sensitivity some low induction of beta-hexosaminidase release was observed with Supercat proteins at the highest concentration of 100 ng/ml. In this patient the allergen mix induced strong basophil activation at all tested concentrations (FIG. 7).

Together the results of IgE reactivity testing and assessment of allergenic activity of SuperCat proteins in comparison with the wildtype allergen mix showed a comparably high reduction of IgE reactivity and allergenic activity of the Supercat proteins. One can therefore expect that cat allergic patients will tolerate the administration of much higher amounts of Supercat proteins in the course of allergen-specific immunotherapy (AIT) which will allow vaccination with fewer and higher doses as compared to allergen-immunotherapy vaccines based on natural wildtype allergens. This will make AIT with Supercat proteins safer and more convenient for the patients.

### Example 5: Antisera Obtained by Immunization with SuperCats 1 to 5 Inhibit Allergic Patients IgE Binding to Cat Allergens

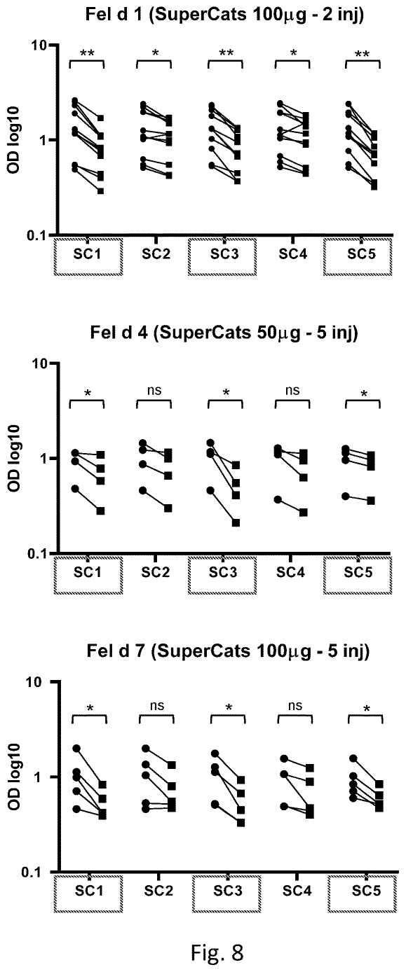

In order to investigate the inhibitory effect of blocking IgG induced by vaccination with SuperCats 1-5, a competitive ELISA was performed as described by Focke-Tejkl M et al. (J Allergy Clin Immunol. 135(2015):1207-17). For this purpose, plates were coated with 1 μg/ml rFel d 1, rFel d 4, or rFel d 7, then they were incubated with pre-immune sera and specific rabbit antisera obtained 4 weeks after the second or fifth injection at a 1:20 dilution. Thereafter, plates were incubated with sera from allergic patients at a dilution of 1:10. The difference in binding of IgE from patient's sera with different allergens after incubation with pre-immune and specific immune sera is shown in FIG. 8.

Reduction of cat allergic patients IgE reactivity to cat allergens (top of FIG. 8) after pre-incubation with rabbit pre-immune and immune sera raised against SuperCats (SC1-SC5) (x-axes) obtained 4 weeks after 2nd injection for Fel d 1 and four weeks after 5th injection for Fel d 4 and 7. Experiments were performed with 11 Fel d 1-allergic patients, and 4 Fel d 4—or Fel d 7-allergic patients. Allergen-specific IgE binding is expressed as OD values on a logarithmic scale (y-axes). Dots indicate ELISA results for IgE binding after pre-incubation with pre-immune rabbit sera, squares indicate the results after pre-incubation with immune sera, and paired values are connected by lines. Differences between groups were compared by Student's dependent t-test for paired samples. Abbreviations: SC1-5—SuperCats 1-5

Antisera raised against SuperCats 1 to 5 (100 μg/injection) showed a significant inhibition of allergic patients IgE binding to Fel d 1 after the second injection. Blocking of IgE binding to Fel d 1 was slightly better with antisera specific for SuperCats 1, 3 and 5 as compared to antisera raised against SupeCats 2 and 4 (FIG. 8). Likewise, antisera raised against SuperCats 1, 3, or 5 (50 μg or 100 μg/injection) showed slightly better blocking of allergic patients IgE binding to Fel d 4 and 7 than antisera raised against SuperCats 2 and 4 (FIG. 8).

These data clearly show that SuperCats 1 to 5 reduced IgE reactivity and allergenic activity. However, Supercats 1, 3 and 5 were identified as being even superior to SuperCats 2 and 4 regarding the induction of allergen-specific protective antibodies.

### Example 6: SuperCats Showed Better Inhibition of Cat Allergic Patients IgE Binding to Fel d 1 as Compared to Two Previously Described PreS-Based Fel d 1-Specific Vaccines (i.e., PreS-2×PJ, PreS-P1-P5)

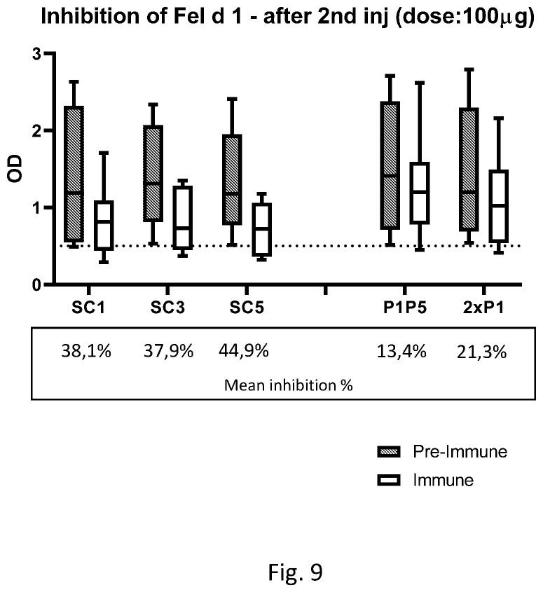

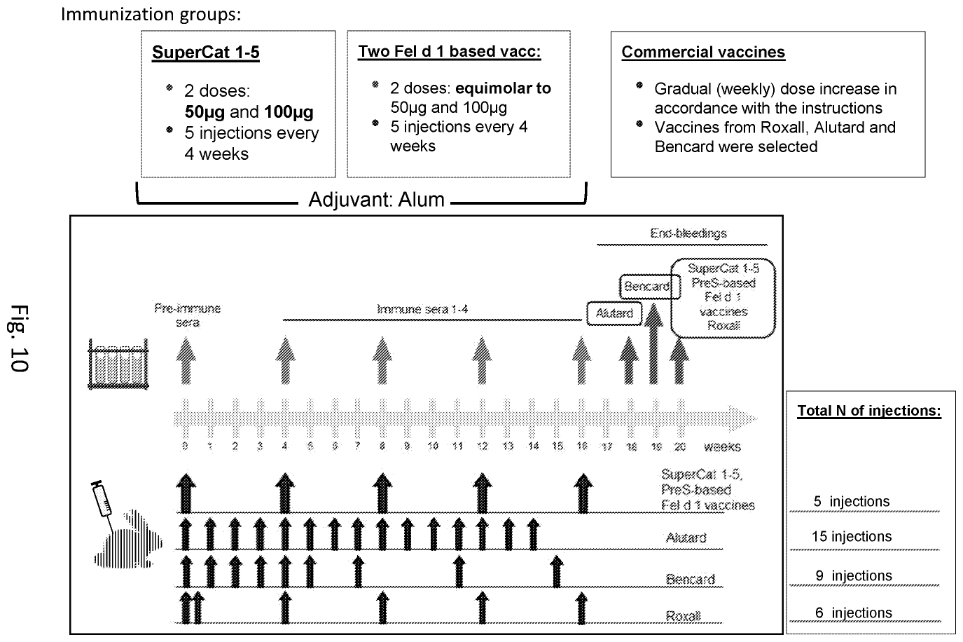

In this example the inhibitory effect of IgG induced by vaccination with two of the previously described PreS-based vaccines for Fel d 1 (i.e., PreS-P1P5 and PreS-2×P1) (Niespodziana K et al. J Allergy Clin Immunol. 127(2011):1562-70) was compared with those induced by vaccination with SuperCats 1, 3, and 5. A competitive ELISA was performed with antisera obtained after identical immunization with equimolar amounts of the vaccines with rabbit antisera samples obtained 4 weeks after 2nd vaccination (FIG. 9).

Reduction of specific IgE reactivity of cat allergic patients (n=11) IgE binding to Fel d 1 after pre-incubation with rabbit pre-immune and immune sera obtained 4 weeks after 2nd injection with SuperCats (SC1, 3 and 5) or previous Fel d 1-based vaccines PreS-P1-P5 and PreS-2×P1. OD values (y-axes) correspond to bound IgE and are represented in Box plots, where boxes represent the interquartile range containing 50% of the data and the horizontal lines in the boxes mark the median, lines outside the boxes indicate max and min. The cut-off was 0.5 (abbreviations: P1P5 and 2×P1—previous Fel d 1 based vaccines PreS-P1-P5 and PreS-2×P1; SC1, 3, 5—SuperCats 1, 3, 5).

It was found that antibodies induced after the 2nd injection with SuperCats inhibited allergic patients IgE binding much better than antibodies induced with PreS-P1P5 or PreS-2×P1 vaccines. Mean percentages of inhibitions with SuperCat-induced antibodies were 37.9-44.9% whereas they were 13.4% for PreS-P1-P5 and 21.3% for PreS-2×P1 (FIG. 9).

These results obtained by a direct comparison in the same experiment show that Supercats, in particular Supercats 1, 3 and 5, are superior to two of the previously described PreS-based vaccines for Fel d 1 (i.e., PreS-P1P5 and PreS-2×P1) regarding the induction of protective antibodies.

### Example 7: SuperCats 1, 3, or 5 are Far Superior to Commercial Allergen Extract Based Vaccines Regarding the Induction of Fel d 4 and Fel d 7-Specific IgG Antibodies

SuperCats 1, 3 and 5 were compared regarding the induction of IgG antibodies against Fel d 4 and Fel d 7 with commercial vaccines from different companies which are available for AIT of cat allergy in Europe (i.e., Alutard, ALK-Abelló; Bencard, Allergie GmbH and Roxall CLUSTOID®) and the two previously described PreS-based Fel d 1 vaccines (i.e., PreS-P1-P5 and PreS-2×P1). Fel d 4 and Fel d 7-specific IgG levels were measured by ELISA in rabbits which had been immunized according to the manufacturer instructions for the commercial vaccines and five times in monthly intervals for SuperCats (FIG. 10). Pre-immune serum samples were obtained shortly before the first immunization, immune samples were collected monthly during immunizations (samples 1-4) and 4 weeks after the last injection for each vaccine (i.e., last samples).

It was found that SuperCats 1, 3 and 5 induced high levels of Fel d 4 and Fel d 7-specific IgG antibodies whereas Alutard and Roxall induced only low levels of Fel d 4 and Fel d 7-specific IgG levels and no induction of Fel d 4 and Fel d 7-specific IgG was observed after vaccination with Bencard and previously described PreS-based Fel d 1 vaccines (i.e., PreS-P1-P5 and PreS-2×P1).

### Example 8: SuperCats 1, 3, or 5 are Far Superior to Commercial Allergen Extract Based Vaccines Regarding the Induction of Fel d 4 and Fel d 7-Specific IgG Antibodies which Block Allergic Patients IgE Binding to Fel d 4 and Fel d 7

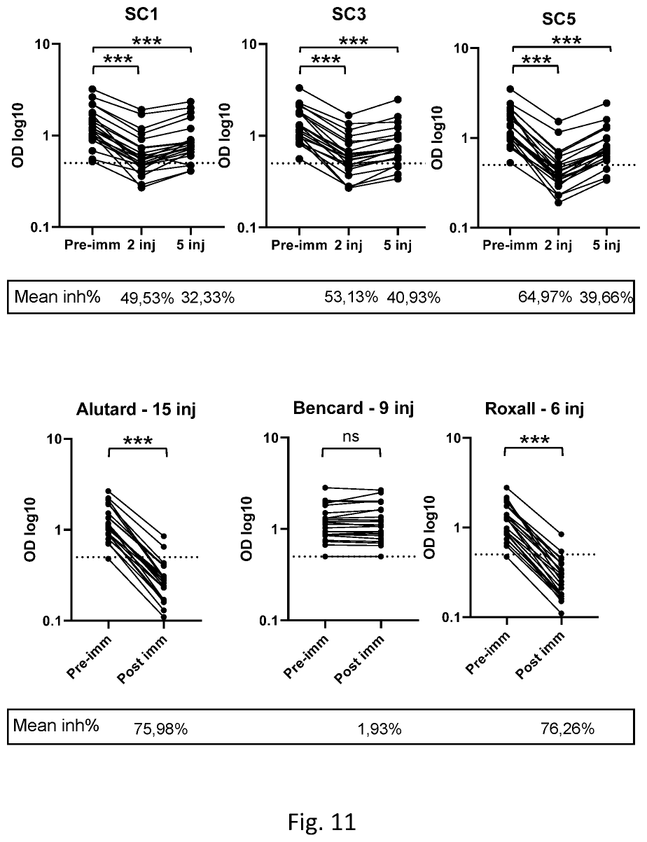

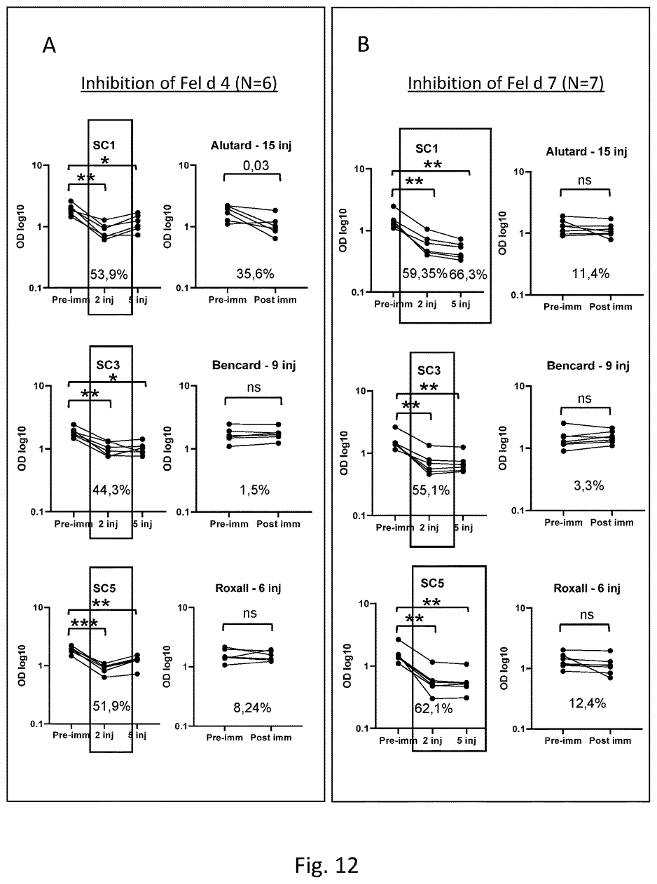

In order to investigate the blocking activity of specific IgGs, IgE binding inhibition experiments were performed with sera from allergic subjects as described (Niespodziana K et al. J Allergy Clin Immunol. 127(2011):1562-70). ELISA plates were coated with 1 μg/ml of each of the allergens; then pre-incubated with rabbit sera obtained before and 4 weeks after the 2nd or 5th injection of SuperCats (100 μg per injection). Inhibition experiments performed with antisera obtained with allergen extract were done with immune sera obtained 4 weeks after last injection which was after 15, 9 and 6 injections for Alutard, Bencard, and Roxall, respectively. The inhibition of allergic patients IgE binding to Fel d 1, Fel d 4 and Fel d 7 after pre-incubation with specific rabbit antisera is shown is shown in FIGS. 11 and 12. Surprisingly, the strongest inhibition of IgE binding to Fel d 1 with SuperCats 1, 3 and 5 was observed after the second injection. The inhibition after the second injection of SuperCats was almost as good as after 6 and 15 injections of Roxall and Alutard, respectively (FIG. 11) whereas Bencard did not induce any relevant blocking antibodies even after 9 injections (FIG. 11).

In FIG. 11 OD values (y-axes) corresponding to allergic patients (n=22) IgE binding to Fel d 1 after pre-incubation of Fel d 1 with Pre-immune sera and sera obtained after the second and fifth injection with SuperCats and after a full vaccination course with allergen extract-based vaccines (i.e., 15, 9 or 6 injections of Alutard, Bencard or Roxall, respectively) are shown. The dots indicate IgE levels (i.e., pair values are connected by a line). Mean % of inhibition are indicated below. Differences between groups were compared by Student's dependent t-test for paired samples (p-values<0.05 were considered significant. *—p value<0.01, **—p value≤0.001, ***—p value≤0.0001; ns—non statistical; abbreviation: SC—SuperCat).

Regarding Fel d 4 and Fel d 7 only a modest inhibition of IgE reactivity to Fel d 4 was observed after 15 injections with Alutard whereas no relevant inhibition of IgE binding to Fel d 4 and Fel d 7 was found for any of the other allergen extract-based vaccines (FIG. 12). Only Supercats 1, 3 and 5 induced strong inhibition of patients IgE binding to Fel d 4 and Fel d 7 over 40% and again inhibition was best after 2 injections suggesting that fewer injections of SuperCats may be necessary to build up protective IgG antibody responses in patients (FIG. 12).

FIG. 12 shows OD values (y-axes) corresponding to allergic patients (n=22) IgE binding to Fel d 1 after pre-incubation of Fel d 4 (left) and Fel d 7 (right) with Pre-immune sera and sera obtained after the second and fifth injection with SuperCats and after a full vaccination course with allergen extract-based vaccines (i.e., 15, 9 or 6 injections of Alutard, Bencard or Roxall, respectively). The dots indicate IgE levels (i.e., pair values are connected by a line). Mean % of inhibition are indicated below. Differences between groups were compared by Student's dependent t-test for paired samples (p-values<0.05 were considered significant. *—p value<0.01, **—p value≤0.001, ***—p value≤0.0001; ns—non statistical; abbreviation: SC—SuperCat).

1. A peptide consisting of 20 to 30 amino acid residues derived from amino acid position 120 to 171 of mature allergen Fel d 4.

2. The peptide of claim 1, wherein the peptide is derived from amino acid position 130 to 171, preferably from amino acid position 140 to 171, more preferably from amino acid position 145 to 171, more preferably from amino acid position 146 to 171, of mature Fel d 4.

3. The peptide of claim 1, wherein Fel d 4 comprises or consists of SEQ ID No: 1.

4. The peptide of claim 1, wherein the peptide comprises or consists of amino acid sequence SEQ ID No: 3.

5. A fusion protein or a conjugate comprising at least one peptide according to claim 1.

6. The fusion protein or conjugate of claim 5, wherein the at least one peptide is conjugated or fused to at least one allergen fragment and/or at least one carrier protein.

7. The fusion protein or conjugate of claim 6, wherein the at least one allergen fragment is derived from at least one furry animal allergen, preferably from at least one cat allergen.

8. The fusion protein or conjugate of claim 7, wherein the at least one cat allergen is selected from the group consisting of Fel d 1 chain 1, Fel d 1 chain 2, Fel d 2, Fel d 3, Fel d 5, Fel d 6, Fel d 7 and Fel d 8, preferably from the group consisting of Fel d 1 chain 1, Fel d 1 chain 2, and Fel d 7, more preferably from the group consisting of Fel d 1 chain 1, Fel d 1 chain 2, and Fel d 7.

9. The fusion protein or conjugate of claim 6, wherein the at least one allergen fragment comprises or consists of 20 to 40 amino acid residues.

10. The fusion protein or conjugate of claim 8, wherein the at least one allergen fragment of the at least one cat allergen is derived from and comprises the N-terminal end or the C-terminal end of said at least one cat allergen.

11. The fusion protein or conjugate of claim 8, wherein the at least one allergen fragment derived from Fel d 1 chain 1 comprises or consists of amino acid residues 1 to 34 of mature Fel d 1 chain 1.

12. The fusion protein or conjugate of claim 8, wherein the at least one allergen fragment derived from Fel d 1 chain 2 comprises or consists of amino acid residues 81 to 109 of mature Fel d 1 chain 2.

13. The fusion protein or conjugate of claim 8, wherein the at least one allergen fragment derived from Fel d 7 comprises or consists of amino acid residues 61 to 97 and/or amino acid residues 124 to 162 of mature Fel d 7.

14. The fusion protein or conjugate of claim 6, wherein the carrier protein is a viral protein or a fragment thereof consisting of 50 to 300, preferably 60 to 250, more preferably 80 to 200, more preferably 100 to 200, amino acid residues.

15. The fusion protein or conjugate of claim 14, wherein the viral protein is a capsid protein.

16. The fusion protein or conjugate of claim 14, wherein the viral protein is derived from a virus of the hepadnaviridae family.

17. The fusion protein or conjugate of claim 16, wherein the virus of the hepadnaviridae family is a Hepatitis B virus.

18. The fusion protein or conjugate of claim 17, wherein the viral protein of the Hepatitis B virus is PreS, PreS1 or PreS2.

19. The fusion protein or conjugate of claim 6, wherein the carrier protein comprises or consists of amino acid sequence SEQ ID No: 4.

20. The fusion protein or conjugate of, wherein the fusion protein comprises at least two peptides according to claim 1.

21. The fusion protein or conjugate of claim 6, wherein two to eight, preferably two to six, of the at least one peptide and two to eight, preferably two to six, of at least one allergen fragment are fused to the N-terminal end and the C-terminal end of the at least one carrier protein.

22. The fusion protein or conjugate of claim 20, wherein two peptides of the at least one peptide are located adjacent to each other within the fusion protein.

23. The fusion protein or conjugate of claim 6, wherein the fusion protein comprises two peptides comprising or consisting of amino acid residues 146 to 171 of mature Fel d 4, two allergen fragments comprising or consisting of amino acid residues 1 to 34 of mature Fel d 1 chain 1, two allergen fragments comprising or consisting of amino acid residues 81 to 109 of mature Fel d 1 chain 2, two allergen fragments comprising or consisting of amino acid residues 81 to 109 of mature Fel d 1 chain 2, two allergen fragments comprising or consisting of amino acid residues 61 to 97 of mature Fel d 7 and two allergen fragments comprising or consisting of amino acid residues 124 to 162 of mature Fel d 7.

24. The fusion protein or conjugate of claim 6, wherein said fusion protein comprises or consists of amino acid sequence SEQ ID No: 5, SEQ ID No: 6, SEQ ID No: 7, SEQ ID No: 8, SEQ ID No: 9, SEQ ID No: 10, SEQ ID No: 11, SEQ ID No: 12, SEQ ID No: 13 or SEQ ID No: 14, preferably SEQ ID No: 5, SEQ ID No: 7, SEQ ID No: 9, SEQ ID No: 11 or SEQ ID No: 13.

25-26. (canceled)

27. Nucleic acid molecule encoding a peptide according to claim 1.

28. Vector comprising a nucleic acid molecule according to claim 27.

29. Host cell comprising a nucleic acid molecule according to claim 27.

30. Vaccine formulation comprising a peptide according to claim 1.

31. Vaccine formulation according to claim 30 for the use in the prevention or treatment of a furry animal allergy, preferably a cat, dog and/or horse allergy.

32. Vaccine formulation according to claim 30, wherein said formulation comprises 10 ng to 1 g, preferably 100 ng to 10 mg, especially 0.5 μg to 200 μg of said fusion protein, nucleic acid molecule or vector.

33. Vaccine formulation according to claim 30, wherein said formulation further comprises at least one adjuvant, pharmaceutical acceptable excipient and/or preservative.


SEQUENCE LISTING (24 sequences)

SEQ ID NO: 1 - protein, 171 aa, Felis catus
HEEENVVRSNIDISKISGEWYSILLASDVKEKIEENGSMRVFVKHIKALDNSSLSFVFHTKENGKCTEIFLVADKTKDGVYTVVYDGYNVFSIVETVYDEYILLHLLNFDKTRPFQLVEFYAREPDVSQKLKEKFVKYCQEHGIVNILDLTEVDRCLQARGSEVAQDSSVE

SEQ ID NO: 2 - protein, 186 aa, Felis catus
MKLLLLCLGLILVCAHEEENVVRSNIDISKISGEWYSILLASDVKEKIEENGSMRVFVEHIKALDNSSLSFVFHTKENGKCTEIFLVADKTKDGVYTVVYDGYNVFSIVETVYDEYILLHLLNFDKTRPFQLVEFYAREPDVSQKLKEKFVKYCQEHGIVNILDLTEVDRCLQARGSEVAQDSSVE

SEQ ID NO: 3 - protein, 26 aa, synthetic construct
  source 1..26: note = Fel d 4 fragment
NILDLTEVDRCLQARGSEVAQDSSVE

SEQ ID NO: 4 - protein, 173 aa, Hepatitis B virus
GGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTN

SEQ ID NO: 5 - protein, 501 aa, synthetic construct
  source 1..501: note = Fusion protein
EICPAVKRDVDLFLTGTPDEYVEQVAQYKALPVVEICPAVKRDVDLFLTGTPDEYVEQVAQYKALPVVNILDLTEVDRCLQARGSEVAQDSSVENILDLTEVDRCLQARGSEVAQDSSVEKEVELILEKTSEPKKYTTYGGKRVVYIEPTEVKDHYKEVELILEKTSEPKKYTTYGGKRVVYIEPTEVKDHYGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNMTTISSSKDCMGEAVQNTVEDLKLNTLGRMTTISSSKDCMGEAVQNTVEDLKLNTLGREEALENFREFLRAKGFNQEIFSPKQSDTCPPGTDQEPEVEEALENFREFLRAKGFNQEIFSPKQSDTCPPGTDQEPEV

SEQ ID NO: 6 - protein, 507 aa, synthetic construct
  source 1..507: note = Fusion protein
EICPAVKRDVDLFLTGTPDEYVEQVAQYKALPVVEICPAVKRDVDLFLTGTPDEYVEQVAQYKALPVVNILDLTEVDRCLQARGSEVAQDSSVENILDLTEVDRCLQARGSEVAQDSSVEKEVELILEKTSEPKKYTTYGGKRVVYIEPTEVKDHYKEVELILEKTSEPKKYTTYGGKRVVYIEPTEVKDHYGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNMTTISSSKDCMGEAVQNTVEDLKLNTLGRMTTISSSKDCMGEAVQNTVEDLKLNTLGREEALENFREFLRAKGFNQEIFSPKQSDTCPPGTDQEPEVEEALENFREFLRAKGFNQEIFSPKQSDTCPPGTDQEPEVHHHHHH

SEQ ID NO: 7 - protein, 501 aa, synthetic construct
  source 1..501: note = Fusion protein
EICPAVKRDVDLFLTGTPDEYVEQVAQYKALPVVMTTISSSKDCMGEAVQNTVEDLKLNTLGRNILDLTEVDRCLQARGSEVAQDSSVENILDLTEVDRCLQARGSEVAQDSSVEKEVELILEKTSEPKKYTTYGGKRVVYIEPTEVKDHYKEVELILEKTSEPKKYTTYGGKRVVYIEPTEVKDHYGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNEEALENFREFLRAKGFNQEIFSPKQSDTCPPGTDQEPEVEEALENFREFLRAKGFNQEIFSPKQSDTCPPGTDQEPEVEICPAVKRDVDLFLTGTPDEYVEQVAQYKALPVVMTTISSSKDCMGEAVQNTVEDLKLNTLGR

SEQ ID NO: 8 - protein, 507 aa, synthetic construct
  source 1..507: note = Fusion protein
EICPAVKRDVDLFLTGTPDEYVEQVAQYKALPVVMTTISSSKDCMGEAVQNTVEDLKLNTLGRNILDLTEVDRCLQARGSEVAQDSSVENILDLTEVDRCLQARGSEVAQDSSVEKEVELILEKTSEPKKYTTYGGKRVVYIEPTEVKDHYKEVELILEKTSEPKKYTTYGGKRVVYIEPTEVKDHYGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNEEALENFREFLRAKGFNQEIFSPKQSDTCPPGTDQEPEVEEALENFREFLRAKGFNQEIFSPKQSDTCPPGTDQEPEVEICPAVKRDVDLFLTGTPDEYVEQVAQYKALPVVMTTISSSKDCMGEAVQNTVEDLKLNTLGRHHHHHH

SEQ ID NO: 9 - protein, 501 aa, synthetic construct
  source 1..501: note = Fusion protein
EICPAVKRDVDLFLTGTPDEYVEQVAQYKALPVVMTTISSSKDCMGEAVQNTVEDLKLNTLGRNILDLTEVDRCLQARGSEVAQDSSVENILDLTEVDRCLQARGSEVAQDSSVEKEVELILEKTSEPKKYTTYGGKRVVYIEPTEVKDHYEEALENFREFLRAKGFNQEIFSPKQSDTCPPGTDQEPEVGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNEEALENFREFLRAKGFNQEIFSPKQSDTCPPGTDQEPEVKEVELILEKTSEPKKYTTYGGKRVVYIEPTEVKDHYMTTISSSKDCMGEAVQNTVEDLKLNTLGREICPAVKRDVDLFLTGTPDEYVEQVAQYKALPVV

SEQ ID NO: 10 - protein, 507 aa, synthetic construct
  source 1..507: note = Fusion protein
EICPAVKRDVDLFLTGTPDEYVEQVAQYKALPVVMTTISSSKDCMGEAVQNTVEDLKLNTLGRNILDLTEVDRCLQARGSEVAQDSSVENILDLTEVDRCLQARGSEVAQDSSVEKEVELILEKTSEPKKYTTYGGKRVVYIEPTEVKDHYEEALENFREFLRAKGFNQEIFSPKQSDTCPPGTDQEPEVGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNEEALENFREFLRAKGFNQEIFSPKQSDTCPPGTDQEPEVKEVELILEKTSEPKKYTTYGGKRVVYIEPTEVKDHYMTTISSSKDCMGEAVQNTVEDLKLNTLGREICPAVKRDVDLFLTGTPDEYVEQVAQYKALPVVHHHHHH

SEQ ID NO: 11 - protein, 501 aa, synthetic construct
  source 1..501: note = Fusion protein
MTTISSSKDCMGEAVQNTVEDLKLNTLGRKEVELILEKTSEPKKYTTYGGKRVVYIEPTEVKDHYNILDLTEVDRCLQARGSEVAQDSSVEEEALENFREFLRAKGFNQEIFSPKQSDTCPPGTDQEPEVEICPAVKRDVDLFLTGTPDEYVEQVAQYKALPVVGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNMTTISSSKDCMGEAVQNTVEDLKLNTLGRKEVELILEKTSEPKKYTTYGGKRVVYIEPTEVKDHYNILDLTEVDRCLQARGSEVAQDSSVEEEALENFREFLRAKGFNQEIFSPKQSDTCPPGTDQEPEVEICPAVKRDVDLFLTGTPDEYVEQVAQYKALPVV

SEQ ID NO: 12 - protein, 507 aa, synthetic construct
  source 1..507: note = Fusion protein
MTTISSSKDCMGEAVQNTVEDLKLNTLGRKEVELILEKTSEPKKYTTYGGKRVVYIEPTEVKDHYNILDLTEVDRCLQARGSEVAQDSSVEEEALENFREFLRAKGFNQEIFSPKQSDTCPPGTDQEPEVEICPAVKRDVDLFLTGTPDEYVEQVAQYKALPVVGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNMTTISSSKDCMGEAVQNTVEDLKLNTLGRKEVELILEKTSEPKKYTTYGGKRVVYIEPTEVKDHYNILDLTEVDRCLQARGSEVAQDSSVEEEALENFREFLRAKGFNQEIFSPKQSDTCPPGTDQEPEVEICPAVKRDVDLFLTGTPDEYVEQVAQYKALPVVHHHHHH

SEQ ID NO: 13 - protein, 501 aa, synthetic construct
  source 1..501: note = Fusion protein
KEVELILEKTSEPKKYTTYGGKRVVYIEPTEVKDHYEEALENFREFLRAKGFNQEIFSPKQSDTCPPGTDQEPEVKEVELILEKTSEPKKYTTYGGKRVVYIEPTEVKDHYEEALENFREFLRAKGFNQEIFSPKQSDTCPPGTDQEPEVNILDLTEVDRCLQARGSEVAQDSSVENILDLTEVDRCLQARGSEVAQDSSVEGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNEICPAVKRDVDLFLTGTPDEYVEQVAQYKALPVVMTTISSSKDCMGEAVQNTVEDLKLNTLGREICPAVKRDVDLFLTGTPDEYVEQVAQYKALPVVMTTISSSKDCMGEAVQNTVEDLKLNTLGR

SEQ ID NO: 14 - protein, 507 aa, synthetic construct
  source 1..507: note = Fusion protein
KEVELILEKTSEPKKYTTYGGKRVVYIEPTEVKDHYEEALENFREFLRAKGFNQEIFSPKQSDTCPPGTDQEPEVKEVELILEKTSEPKKYTTYGGKRVVYIEPTEVKDHYEEALENFREFLRAKGFNQEIFSPKQSDTCPPGTDQEPEVNILDLTEVDRCLQARGSEVAQDSSVENILDLTEVDRCLQARGSEVAQDSSVEGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNEICPAVKRDVDLFLTGTPDEYVEQVAQYKALPVVMTTISSSKDCMGEAVQNTVEDLKLNTLGREICPAVKRDVDLFLTGTPDEYVEQVAQYKALPVVMTTISSSKDCMGEAVQNTVEDLKLNTLGRHHHHHH

SEQ ID NO: 15 - protein, 34 aa, synthetic construct
  source 1..34: note = Fel d 4 fragment
EEENVVRSNIDISKISGEWYSILLASDVKEKIEE

SEQ ID NO: 16 - protein, 43 aa, synthetic construct
  source 1..43: note = Fel d 4 fragment
HIKALDNSSLSFVFHTKENGKCTEIFLVADKTKDGVYTVVYDG

SEQ ID NO: 17 - protein, 35 aa, synthetic construct
  source 1..35: note = Fel d 4 fragment
HLLNFDKTRPFQLVEFYAREPDVSQKLKEKFVKYC

SEQ ID NO: 18 - protein, 37 aa, synthetic construct
  source 1..37: note = Fel d 7 fragment
QDPPASGEDTMAMSGKWYLKAMITDRETSWKKPELVT

SEQ ID NO: 19 - protein, 35 aa, synthetic construct
  source 1..35: note = Fel d 7 fragment
KKPELVTPMTLTVLEGGNLKAETTLLTNGQCKEVE

SEQ ID NO: 20 - protein, 36 aa, synthetic construct
  source 1..36: note = Fel d 7 fragment
KEVELILEKTSEPKKYTTYGGKRVVYIEPTEVKDHY

SEQ ID NO: 21 - protein, 35 aa, synthetic construct
  source 1..35: note = Fel d 7 fragment
CEGEMQGEQARMAKLVGRDPESNEEALENFREFLR

SEQ ID NO: 22 - protein, 39 aa, synthetic construct
  source 1..39: note = Fel d 7 fragment
EEALENFREFLRAKGFNQEIFSPKQSDTCPPGTDQEPEV

SEQ ID NO: 23 - protein, 34 aa, synthetic construct
  source 1..34: note = Fel d 1 fragment
EICPAVKRDVDLFLTGTPDEYVEQVAQYKALPVV

SEQ ID NO: 24 - protein, 29 aa, synthetic construct
  source 1..29: note = Fel d 1 fragment
MTTISSSKDCMGEAVQNTVEDLKLNTLGR